(19) United States
(12) Patent Application Publication
(10) Pub. No.: US 2003/0220325 A1
(43) Pub. Date: Nov. 27, 2003

(54) Arylsulfone derivatives

(76) Inventors: Ruth E. Tenbrink, Kalamazoo, MI (US); Steven W. Kortum, Kalamazoo, MI (US)

Correspondence Address: FLYNN, THIEL, BOUTELL & TANIS, P.C., 2026 RAMBLING ROAD, KALAMAZOO, MI, 49008-1699, US

(21) Appl. No.: 10/371,215

(22) Filed: Feb. 20, 2003

Related U.S. Application Data
(60) Provisional application No. 60/359,179, filed on Feb. 22, 2002.

(51) Int. Cl. A61K 31/551; A61K 31/53; A61K 31/519; A61K 31/502; C07D 487/02

(52) U.S. Cl. 514/218; 514/242; 514/248; 514/264.1; 514/266.2; 540/575; 544/284; 544/279; 544/184; 544/236

(57) ABSTRACT

The invention provides compounds of the formula

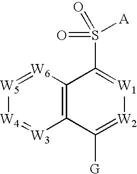

and methods of using those compounds for treating a disease or condition in a mammal wherein a 5-HT receptor, such as a 5-HT₆ receptor, is implicated and modulation of a 5-HT function is desired, wherein A, G and W₁-W₆ are defined as herein.

### CROSS REFERENCE TO RELATED APPLICATION

This application claims the benefit of U.S. provisional application Ser. No. 60/359 179, filed Feb. 22, 2002, under 35 USC 119(e)(i), which is incorporated herein by reference.

### FIELD OF THE INVENTION

The present invention relates to novel arylsulfone derivatives, and more specifically, relates to arylsulfone compounds of formulae I and II described herein below. These compounds are 5-HT receptor ligands and are useful for treating diseases wherein modulation of 5-HT activity is desired.

### BACKGROUND OF THE INVENTION

Serotonin has been implicated in a number of diseases and conditions that originate in the central nervous system. These include diseases and conditions related to sleeping, eating, perceiving pain, controlling body temperature, controlling blood pressure, depression, anxiety, schizophrenia, and other bodily states. Serotonin also plays an important role in peripheral systems, such as the gastrointestinal system, where it has been found to mediate a variety of contractile, secretory, and electrophysiologic effects.

As a result of the broad distribution of serotonin within the body, there is a tremendous interest in drugs that affect serotonergic systems. In particular, agonists, partial agonists and antagonists are of interest for the treatment of a wide range of disorders, including anxiety, depression, hypertension, migraine, obesity, compulsive disorders, schizophrenia, autism, neurodegenerative disorders (e.g. Alzheimer's disease, Parkinsonism, and Huntington's chorea), and chemotherapy-induced vomiting.

The major classes of serotonin receptors (5-HT₁₋₇) contain fourteen to eighteen separate receptors that have been formally classified. See Glennon, et al., Neuroscience and Behavioral Reviews, 1990, 14, 35; and D. Hoyer, et al. Pharmacol. Rev. 1994, 46, 157-203.

There is currently a need for pharmaceutical agents that are useful to treat diseases and conditions that are associated with 5-HT receptors. In particular, there is a need for agents that can selectively bind to individual receptor sub-types (e.g. receptor-specific agonists or antagonists); such agents would be useful as pharmaceutical agents, or would be useful to facilitate the study of the 5-HT receptor family, or to aid in the identification of other compounds that selectively bind to the specific 5-HT receptors.

For example, The 5-HT₆ receptor was identified in 1993 (Monsma et al. Mol. Pharmacol. 1993, 43, 320-327 and Ruat, M. et al. Biochem. Biophys. Res. Com. 1993, 193, 269-276). Several antidepressants and atypical antipsychotics bind to the 5-HT₆ receptor with high affinity and this binding may be a factor in their profile of activities (Roth et al. J. Pharm. Exp. Therapeut. 1994, 268, 1403-1410; Sleight et al. Exp. Opin. Ther. Patents 1998, 8, 1217-1224; Bourson et al. Brit. J. Pharm. 1998, 125, 1562-1566; Boess et al. Mol. Pharmacol. 1998, 54, 577-583; Sleight et al. Brit. J. Pharmacol. 1998, 124, 556-562). In addition, the 5-HT₆ receptor has been linked to generalized stress and anxiety states (Yoshioka et al. Life Sciences 1998, 17/18, 1473-1477). Together these studies and observations suggest that compounds that antagonize the 5-HT₆ receptor will be useful in treating disorders of the central nervous system.

### INFORMATION DISCLOSURE

GB 2 321 457 discloses prostaglandin synthase inhibitors that are useful for the treatment of central nervous system diseases and weight problems.

U.S. Pat. No. 6,004,979 discloses compounds having a quinoline ring system that are useful for treating cardiovascular and gastrointestinal problems, asthma and Alzheimer's disease.

WO 92/06683 discloses aryl sulfone derivatives useful for treatment of retroviral disease.

WO 93/24442 discloses naphthalene derivatives that are substituted at C-1 with sulfonyl-benzoic acid and at C-4 with hydrogen. The derivates are useful for treating prostatomegaly and prostate cancer.

### SUMMARY OF THE INVENTION

In one aspect, the invention features compounds of formula I:

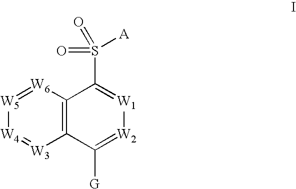

or a pharmaceutically acceptable salt thereof, wherein

Each of W₁-W₆ are independently N or —C(R₁), provided that no more than three of W₁-W₆are simultaneously N, and further provided that when W₁ is N that W₂ is not —CHaryl, or —CHaryl in which the aryl group is substituted with halo, —OH, —CN, —NO₂, —CF₃, —COOR₁, tetrazolyl, or isoxazolyl;

Each R₁ is independently selected from H, halo, alkyl, cycloalkyl, substituted alkyl, —OH, alkoxy, substituted alkoxy, —SH, —S-alkyl, —S-substituted alkyl, —CN, —NO₂, —NR₄R₅, —NR₄SO₂-alkyl, —NR₄SO₂-aryl, —COOR₄, —CONR₄R₅, —SO₂NR₄R₅, —SO₂-alkyl, het, substituted het, aryl, and substituted aryl;

Each R₄ and R₅ is independently H, alkyl, cycloalkyl, substituted alkyl, aryl, het, substituted aryl, or substituted het, or R₄ and R₅ when taken together, along with the atom to which they are bound, form a five, six, or seven-membered ring which contains 1-3 heteroatoms selected from N, O, or S;

A is a five- or six-membered monocyclic aromatic ring; a eight- or ten-membered fused aromatic ring, the five- or six-membered monocyclic aromatic ring and the eight- or ten-membered fused aromatic ring system each optionally containing up to three heteroatoms (O, N, S); or a nine-membered fused aromatic ring system containing one to three heteroatoms (O, N, S), and each of the five- or six-membered monocyclic aromatic ring and the eight- to ten-membered fused aromatic ring systems being optionally substituted with 1-4 of R₁, and when all of W₁-W₆ are —(CH)R₁ A is substituted with at least one electron donating group;

G is a group selected from

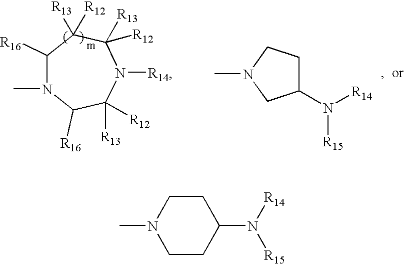

Each R₁₂ and R₁₆ is independently selected from H, alkyl, and oxo, provided that R₁₃ is absent when the oxo moiety is bound to the same carbon;

Each R₁₃ is H or alkyl;

Each R₁₄ and R₁₅ is independently H, alkyl, and substituted alkyl; and

m is 0 or 1.

In another aspect, the invention provides a pharmaceutical composition comprising a therapeutically effective amount of a compound of formula I or a pharmaceutically acceptable salt thereof. The composition may also include a pharmaceutically acceptable carrier.

The present invention further provides a method for treating a disease or condition in a mammal wherein a 5-HT receptor is implicated and modulation of a 5-HT function is desired comprising administering to the mammal a therapeutically effective amount of a compound of formula I, described above, or formula II:

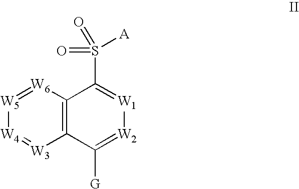

wherein

Each of W₁-W₆ are independently N or —C(R₁), provided that no more than three of W₁-W₆are simultaneously N;

Each R₁ is independently selected from H, halo, alkyl, cycloalkyl, substituted alkyl, —OH, alkoxy, substituted alkoxy, —SH, —S-alkyl, —S-substituted alkyl, —CN, —NO₂, —NR₄R₅, —NR₄SO₂-alkyl, —NR₄SO₂-aryl, —COOR₄, —CONR₄R₅, —SO₂NR₄R₅, —SO₂-alkyl, het, substituted het, aryl, and substituted aryl;

Each R₄ and R₅ is independently H, alkyl, cycloalkyl, substituted alkyl, aryl, het, substituted aryl, or substituted het, or R₄ and R₅ when taken together, along with the atom to which they are bound, form a five, six, or seven-membered ring which contains 1-3 heteroatoms selected from N, O, or S;

A is a five- or six-membered monocyclic aromatic ring; a eight- or ten-membered fused aromatic ring, the five- or six-membered monocyclic aromatic ring and the eight- or ten-membered fused aromatic ring system each optionally containing up to three heteroatoms (O, N, S); or a nine-membered fused aromatic ring system containing one to three heteroatoms (O, N, S), and each of the five- or six-membered monocyclic aromatic ring and the eight- to ten-membered fused aromatic ring systems being optionally substituted with 1-4 of R₁;

G is a group selected from

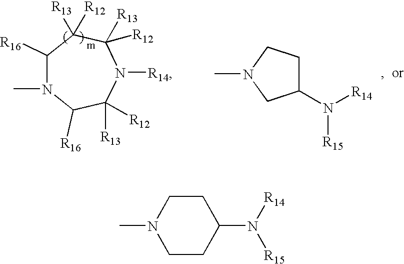

Each R₁₂ and R₁₆ is independently selected from H, alkyl, and oxo, provided that R₁₃ is absent when the oxo moiety is bound to the same carbon;

Each R₁₃ is H or alkyl;

Each R₁₄ and R₁₅ is independently H, alkyl, and substituted alkyl; and

m is 0 or 1.

The present invention further provides a method for treating a disease or condition in a mammal wherein a 5-HT₆ receptor is implicated and modulation of a 5-HT₆ function is desired comprising administering to the mammal a therapeutically effective amount of a compound of formula I or II, or a pharmaceutically acceptable salt thereof.

Embodiments of the invention may include one or more of the following features. Each R₁ is independently selected from H, halo, C₁-C₆ alkyl, C₃-C₇ cycloalkyl, C₁-C₃ alkyl-C₃-C₇-cycloalkyl, —CF₃, —OH, —O—(C₁-C₆-alkyl), —O—C₂-C₆-alkyl—OH, —O—C₂-C₆-alkyl—NR₂R₃, —OCF₃, —SH, —S—(C₁-C₆-alkyl), —CN, —NO₂, —NR₄R₅, —NHSO₂-C₁-C₄-alkyl, —COOR₄, —CONR₄R₅, —SO₂NR₄R₅, —SO₂—C₁-C₄-alkyl, and aryl optionally substituted with 1 to 3 of H, halo, C₁-C₆-alkyl, C₁-C₆-cycloalkyl, —OH, —O—(C₁-C₆-alkyl), —CN, —NR₄R₅, —CONR₄R₅, or —SO₂NR₄R₅. Each R₂ and R₃ is independently H or C₁-C₄-alkyl. Each R₄ and R₅ is independently H, C₁-C₄-alkyl, C₃-C₇-cycloalkyl, or C₁-C₃-alkyl-C₃-C₇-cycloalkyl. Each R₁₂ and R₁₆ is independently selected from H, C₁-C₄-alkyl, and oxo. Each R₁₃ is H or C₁-C₄-alkyl. Each R₁₄ and R₁₅ is independently H, C₁-C₆-alkyl, or C₂-C₄-alkyl—OH. At least one of W₁-W₆ is N. All of W₁-W₆ are —C(R₁). A is phenyl optionally substituted with alkyl. m is 0. R₁₄ is —CH₃. Each R₁₂ is —CH₃. A is substituted with one —CH₃ group. A is substituted with two-CH₃ groups. The compound is 1-[4-(Phenylsulfonyl)-1-naphthyl]piperazine; Cis-3,5-Dimethyl-1-[4-(phenylsulfonyl)-1-naphthyl]piperazine; 1-[4-(Phenylsulfonyl)-1-naphthyl]-1,4-diazepane; 1-{4-[(2,5-Dimethylphenyl)sulfonyl]-1-naphthyl}piperazine; 4-Methylphenyl 4-(1-piperazinyl)-1-naphthyl sulfone; 4-(4-Methyl-1-piperazinyl)-1-naphthyl phenyl sulfone; or a pharmaceutically acceptable salt thereof. G is

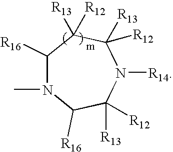

The compounds of formulae I and II also can include isotopic labels. For example the compounds may contain an isotopic label such as at least one atom selected from Carbon-11, Nitrogen-13, Oxygen-15, and Fluorine-18. Isotopically labeled compounds may be used in positron emission tomography, single photon emission computed technology and nuclear magnetic resonance imaging spectroscopy.

Generally, compounds of the present invention are 5-HT ligands. In particular, they can selectively bind to the 5-HT₆ receptor (e.g. receptor-specific agonists or antagonists). Thus, they are useful for treating diseases wherein modulation of 5-HT activity, specifically 5-HT₆ activity, is desired. Therefore, the compounds of this invention are useful for the treatment of diseases or disorders of the central nervous system. More specifically, for the treatment of psychosis, paraphrenia, psychotic depression, mania, schizophrenia, schizophreniform disorders, anxiety, migraine headache, drug addiction, convulsive disorders, personality disorders, post-traumatic stress syndrome, alcoholism, panic attacks, obsessive-compulsive disorders, and sleep disorders. The compounds of this invention are also useful to treat psychotic, affective, vegetative, and psychomotor symptoms of schizophrenia and the extrapyramidal motor side effects of other antipsychotic drugs. This last action will allow higher doses of antipsychotics to be used and thus greater antipsychotic efficacy to be obtained as a result of a reduction in side effects. The compounds of this invention are also useful in the modulation of eating behavior and thus are useful in treating excess weight and associated morbidity and mortality.

The present invention further provides a method for treating or preventing diseases or disorders of the central nervous system comprising administering a therapeutically effective amount of a compound of formula I or II, or a pharmaceutically acceptable salt thereof to the mammal. In particular, compounds of formula I or II are useful in treating depression, schizophrenia, schizophreniform disorder, and schizoaffective disorder. In some embodiments compounds of formula I or II may have activity against other diseases or disorders including, but are not limited to, the following: obesity, delusional disorder, a stress related disease (e.g. general anxiety disorder), panic disorder, a phobia, obsessive compulsive disorder, post-traumatic-stress syndrome, immune system depression, a stress induced problem with the urinary, gastrointestinal or cardiovascular system (e.g., stress incontinence), neurodegenerative disorders, autism, chemotherapy-induced vomiting, hypertension, migraine headaches, cluster headaches, sexual dysfunction in a mammal (e.g. a human), addictive disorder and withdrawal syndrome, an adjustment disorder, an age-associated learning and mental disorder, anorexia nervosa, apathy, an attention-deficit disorder due to general medical conditions, attention-deficit hyperactivity disorder, behavioral disturbance (including agitation in conditions associated with diminished cognition (e.g., dementia, mental retardation or delirium)), bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, movement disorder (e.g., Huntington's disease or Tardive Dyskinesia), oppositional defiant disorder, peripheral neuropathy, post-traumatic stress disorder, premenstrual dysphoric disorder, a psychotic disorder (brief and long duration disorders, psychotic disorder due to medical condition, psychotic disorder NOS), mood disorder (major depressive or bipolar disorder with psychotic features) seasonal affective disorder, a sleep disorder, a specific developmental disorder, agitation disorder, selective serotonin reuptake inhibition (SSRI) “poop out” syndrome or a Tic disorder (e.g., Tourette's syndrome).

The present invention further provides a method for treating anxiety, depression or stress related disorders comprising administering a therapeutically effective amount of a compound of formula I or II, or a pharmaceutically acceptable salt thereof to the mammal.

The present invention further provides isotopically labeled compounds of formulae I or II.

The present invention further provides a method of performing positron emission tomography comprising incorporating an isotopically labeled compound of formulae I or II or a pharmaceutically acceptable salt thereof into tissue of a mammal and detecting the compound distributed into said tissue.

The present invention further provides a method of performing nuclear magnetic resonance imaging comprising:

incorporating an isotopically labeled compound of formulae I or II or a pharmaceutically acceptable salt thereof into tissue of a mammal and detecting the compound distributed in said tissue.

The present invention further provides a method of performing single photon emission computed tomography comprising incorporating an isotopically labeled compound of formula I or II or a pharmaceutically acceptable salt thereof into tissue of a mammal and detecting the compound distributed into said tissue.

The present invention further provides the use of a compound of formulae I and II or a pharmaceutically acceptable salt thereof to prepare a medicament for treating or preventing diseases or disorders of the central nervous system.

The present invention may also provide novel intermediates and processes for preparing compounds of I or II.

### DETAILED DESCRIPTION OF THE INVENTION

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours, “rt” for room temperature, e.g., 18-25° C., and etc.).

The following definitions are used, unless otherwise described.

The carbon atom content of various hydrocarbon-containing moieties can be indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The term “halo” refers to a halogen atom selected from Cl, Br, I, and F.

The term “alkyl” refers to both straight- and branched-chain moieties. Unless otherwise specifically stated alkyl moieties include between 1 and 10 carbon atoms.

The term “alkenyl” refers to both straight- and branched-chain moieties containing at least one —C═C—. Unless otherwise specifically stated alkenyl moieties include between 1 and 10 carbon atoms.

The term “alkynyl” refers to both straight- and branched-chain moieties containing at least one —C≡C—. Unless otherwise specifically stated alkynyl moieties include between 1 and 10 carbon atoms.

The term “alkoxy” refers to —O—alkyl groups.

The term “cycloalkyl” refers to a cyclic alkyl moiety. Unless otherwise specifically stated cycloalkyl moieties will include between 3 and 7 carbon atoms.

The term “cycloalkenyl” refers to a cyclic alkenyl moiety. Unless otherwise specifically stated cycloalkenyl moieties will include between 3 and 7 carbon atoms and at least one —C═C— group within the cyclic ring.

The term “amino” refers to —NH₂.

The term “heterocycloalkyl” refers to a cyclic alkyl moiety including 1-4 heteroatoms in the ring. The heteroatoms are selected from the group consisting of oxygen, sulfur, and nitrogen. Unless otherwise specifically stated heterocycloalkyl moieties include between 5 and 7 ring atoms.

The term “aryl” refers to phenyl and naphthyl.

The term “het” is a C-linked five- (5) membered heteroaryl ring having 1-4 heteroatoms selected from the group consisting of oxygen, sulfur, and nitrogen; a C-linked six (6) membered heteroaryl ring having 1-3 nitrogen atoms; a eight (8) membered bicyclic heteroaryl ring system having 1-3 heteroatoms selected from the group consisting of oxygen, sulfur, and nitrogen; and a ten (10) membered bicyclic heteroaryl ring system having 1-3 heteroatoms selected from the group consisting of oxygen, sulfur, and nitrogen.

Examples of “het” include, but are not limited to, pyridinyl, thiophenyl, furanyl, pyrazolyl, pyrimidinyl, pyridyl, pyridazinyl, imidazolyl, isoxazolyl, pyrazolyl, oxazolyl, oxathiazolyl, oxadiazolyl, thiazolyl, isothiazolyl, thienyl, pyrrolyl, isopyrrolyl, oxathiazolyl-1-oxide, thiadiazoyl, triazolyl, tetrazolyl, thiazolinyl, thiazoledionyl, thiatriazolyl, dithiazolonyl, indoyl, indolinyl, benzofuranyl, benzothiophenyl, benzisoxazolyl, benzimidazoyl, benzoxazolyl, quinolinyl, isoquinolinyl, and quinovalinyl.

The term “substituted alkyl” refers to an alkyl moiety including 1-4 substituents selected from halo, cycloalkyl, cycloalkenyl, heterocycloalkyl, het, aryl, —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, and —NO₂,. Each of the cycloalkyl, heterocycloalkyl, het, aryl, and cycloalkenyl may be optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NO₂, alkyl, substituted alkyl, halo, cycloalkyl, cycloalkenyl, heterocycloalkyl, het, and aryl. The cycloalkyl, cycloalkenyl, heterocycloalkyl, het, and aryl may be optionally substituted with 1-3 substituents selected from halo and Q₁₅.

The term “substituted het” refers to a het moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NO₂, alkyl, substituted alkyl, halo, cycloalkyl, cycloalkenyl, heterocycloalkyl, het, and aryl. The cycloalkyl, cycloalkenyl, heterocycloalkyl, het, and aryl may be optionally substituted with 1-3 substituents selected from halo and Q₁₅.

The term “substituted alkenyl” refers to a alkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NO₂, alkyl, substituted alkyl, halo, cycloalkyl, cycloalkenyl, heterocycloalkyl, het, and aryl. The cycloalkyl, cycloalkenyl, heterocycloalkyl, het, and aryl may be optionally substituted with 1-3 substituents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q_{10, —S(O)}₂NQ₁₀Q₁₀, —NQ₁₀S(O)₂Q₁₀, —NQ₁₀S(O)Q₁₀, —NO₂, alkyl, substituted alkyl, halo, cycloalkyl, heterocycloalkyl, het, aryl, and cycloalkenyl. The cycloalkyl, heterocycloalkyl, het, aryl, and cycloalkenyl may be optionally substituted with 1-3 substituents selected from halo and Q₁₅.

The term “substituted cycloalkenyl” refers to a cycloalkenyl moiety including 1-3 substituents -OQ₁₀, —SQ₁₀, —S(O)₂Q₁₀, —S(O)Q₁₀, —OS(O)₂Q₁₀, —C(═NQ₁₀)Q₁₀, —SC(O)Q₁₀, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NO₂, alkyl, substituted alkyl, halo, cycloalkyl, cycloalkenyl, heterocycloalkyl, het, and aryl. The cycloalkyl, cycloalkenyl, heterocycloalkyl, het, and aryl may be optionally substituted with 1-3 substituents selected from halo and Q₁₅.

Each Q₁₀ is independently selected from —H, alkyl, cycloalkyl, heterocycloalkyl, het, cycloalkenyl, and aryl. The het, heterocycloalkyl, cycloalkyl, cycloalkenyl, and aryl may be optionally substituted with 1-3 substituents selected from halo and Q₁₃.

Each Q₁₁ is independently selected from —H, halo, alkyl, aryl, and cycloalkyl. The alkyl and cycloalkyl may be optionally substituted with 1-3 substituents independently selected from halo, —NO₂, —CN, ═S, ═O, and Q₁₄. The aryl may be optionally substituted with 1-3 substituents independently selected from halo, —NO₂, —CN, and Q₁₄.

Each Q₁₃ is independently selected from Q₁₁—OQ₁₁, —SQ₁₁, —S(O)₂Q₁₁, —S(O)Q₁₁, —OS(O)₂Q₁₁, —C(═NQ₁₁)Q₁₁, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(O)C(Q₁₆)₂OC(O)Q₁₀, —CN, ═O, ═S, —NQ₁₁C(O)Q₁₁, —NQ₁₁C(O)NQ₁₁Q₁₁, —S(O)₂NQ₁₁Q₁₁, —NQ₁₁S(O)₂Q₁₁, —NQ₁₁S(O)Q₁₁, and —NO₂, provided that Q₁₃ is not ═O or ═S when Q₁₀ is aryl or het.

Each Q₁₄ is —H or a substituent selected from alkyl, cycloalkyl, cycloalkenyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —CN, —NQ₁₆C(O)Q₁₆, —NQ₁₆C(O)NQ₁₆Q₁₆, —S(O)₂NQ₁₆Q₁₆, and —NQ₁₆S(O)₂Q₁₆. The alkyl, cycloalkyl, and cycloalkenyl may be further substituted with ═O or 50 S.

Each Q₁₅ is alkyl, cycloalkyl, cycloalkenyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(O)C(Q₁₆)₂OC(O)Q₁₆, —CN, —NQ₁₆C(O)Q₁₆, —NQ₁₆C(O)NQ₁₆Q₁₆, —S(O)₂NQ₁₆Q₁₆, —NQ₁₆S(O)₂Q₁₆, —NQ₁₆S(O)Q₁₆, and —NO₂. The alkyl, cycloalkyl, and cycloalkenyl may be further substituted with ═O or ═S.

Each Q₁₆ iS independently selected from —H, alkyl, and cycloalkyl. The alkyl and cycloalkyl may be optionally substituted with 1-3 halos.

Mammal denotes human and animals.

The term “electron donating group” refers to the ability of a substituent to donate electrons relative to that of hydrogen if the hydrogen atom occupied the same position on the molecule. The term “electron donating group” is well understood by one skilled in the art and is discussed in Advanced Organic Chemistry by J. March, John Wiley & Sons, New York, N.Y., pp. 16-18 (1985) and the discussion therein is incorporated herein by reference. Electron donating groups include such groups as hydroxy, lower alkoxy, including methoxy, ethoxy and the like; amino, lower alkylamino; di(loweralkylamino); aryloxy, such as phenoxy, mercapto, lower alkythio, lower alkylmercapto, and the like. The term “lower alkyl” refers to a C₁-C₄-alkyl.

It is to be understood that the present invention encompasses any racemic, optically-active, polymorphic, tautomeric, or stereoisomeric form, or mixture thereof, of a compound of the invention, which possesses the useful properties described herein.

In cases where compounds are sufficiently basic or acidic to form stable nontoxic acid or base salts, administration of the compounds as pharmaceutically acceptable salts may be appropriate. Examples of pharmaceutically acceptable salts which are within the scope of the present invention include organic acid addition salts formed with acids which form a physiological acceptable anion and inorganic salts. Examples of pharmaceutically acceptable salts include, but are not limited to, the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethane-sulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic and toluenesulfonic.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Although the following schemes include compounds in which all of W1-W6 are —CR₁, compounds having one or more of W₁-W₆ being a nitrogen atom can be produced via similar schemes utilizing appropriate starting materials. All starting materials are commercially available or can be made by procedures well known to those skilled in the art.

Chart A depicts the synthesis of sulfones (5). Commercially available arene (1) is sulfonated using chlorosulfonic acid, sulfuric acid, or SO₃ either neat or in solvents such as dichloromethane, chloroform, carbon tetrachloride, or dichloroethane between the temperatures of −78° C. and 85° C., to give sulfonic acid (2). Sulfonic acid (2) is converted to sulfonyl chloride (3) using thionyl chloride, PCl₅, PCl₃, or other chlorinating agents such as are discussed in or referred to in March, Advanced Organic Chemistry-Reactions, Mechanisms and Structures, 4th Ed., 1992. Sulfonyl chloride (3) be may synthesized directly from (1) using chlorosulfonic acid in solvents such as dichloromethane, chloroform, carbon tetrachloride, dichloroethane between the temperatures of −78° C. and 85° C., or using thionyl chloride in the presence of sulfuric acid.

Sulfonyl halide (3) is treated with aryl (6) in the presence of a Friedel-Crafts reagent such as AlCl₃, AlBr₃, FeCl₃, SnCl₄, BCl₃, BF₃, H₂SO₄, ZnCl₂, polyphosphoric acid, or other reagent known to those well-versed in the art in solvents such as nitromethane, nitrobenzene, or carbon disulfide at temperatures between 0° C. and 200° C. to give sulfone (7). Alternatively, sulfone (7) may be synthesized directly from naphthalene (1) and aryl sulfonyl halide (4) or aryl sulfonic acid (5) in the presence of a Friedel-Crafts reagent such as AlCl₃, AlBr₃, FeCl₃, SnCl₄, BCl₃, BF₃, H₂SO₄, ZnCl₂, polyphosphoric acid, or other reagent known to those well-versed in the art in solvents such as nitromethane, nitrobenzene, or carbon disulfide at temperatures between 0° C. and 200° C.

Chart B shows an alternative route to sulfone (7) and sulfonyl halide (3). Aniline (9) is commercially available or is prepared from nitro (8) by reduction using Raney nickel and hydrazine or Pd or Pt catalysts and hydrogen. Nitro (8) is itself prepared by nitration of arene (1) using HNO₃/H₂SO₄ or other methods well known to those versed in the art. Aniline (9) is then treated with sodium nitrite in a strong acid such as aqueous sulfuric acid, or with butyl nitrite in acetic acid or trifluoroacetic acid, and then with thiophenol (10) at −30° C. to room temperature to give solids, which are collected and then oxidized to sulfone (7) using oxidants such as m-chloroperoxybenzoic acid, peracetic acid, hydrogen peroxide, sodium tunstate, and Oxone, iodobenzene dichloride, sodium periodate, t-butylhypochlorite, and potassium permanganate in solvents such as dichloromethane, chloroform, acetic acid, water at temperatures ranging from room temperature to 120° C., to give sulfone (7). Alternatively, aniline (9) is treated with conc. HCl and sodium nitrite, followed by SO₂ and CuCl₂-2H₂O in acetic acid at temperatures ranging from 0° C. to 120° C. to give sulfonyl halide (3).

Chart C depicts the synthesis of sulfone amine (13). Sulfone (7) is treated with a cyclic diamine (11) in the presence of a base such as triethyl amine, diisopropyl amine, potassium carbonate, or other bases known to those well-versed in the art in solvents such as pyridine, acetonitrile, dimethylformamide, alcoholic solvents such as ethanol or isopropanol, ethyl acetate, and dichloromethane at temperatures ranging from room temperature to 200° C., to give protected sulfone amine (12) when Y is a protecting group such as Boc, Cbz, Fmoc, tert-butyl, or acyl, or sulfone amine (13) when Y is hydrogen or alkyl. When Y is a protecting group it may be removed by methods well-known to those versed in the art (see, for example, Green and Wuts, “Protective Groups in Organic Synthesis,” 3rd Ed., Wiley Interscience) to give sulfone amine (13).

In each of charts A-C shown below, X₁, typically is halo or -Otf, X₂ and X₃, typically are halo, Y, typically is a protecting group for nitrogen, and Aryl, typically is a 5- or 6-membered aromatic ring which may contain one or more heteroatoms, e.g., O, N, or S. Protecting groups for nitrogen include, but are not limited to, carbobenzyloxy (CBz), 1,1 dimethylcarbamate, tert butoxy carbonyl (BOC) and the like. Examples of other suitable protecting groups are known to person skilled in the art and can be found in “Protective Groups in Organic synthesis,” 3rd Edition, authored by Theodora Greene and Peter Wuts.

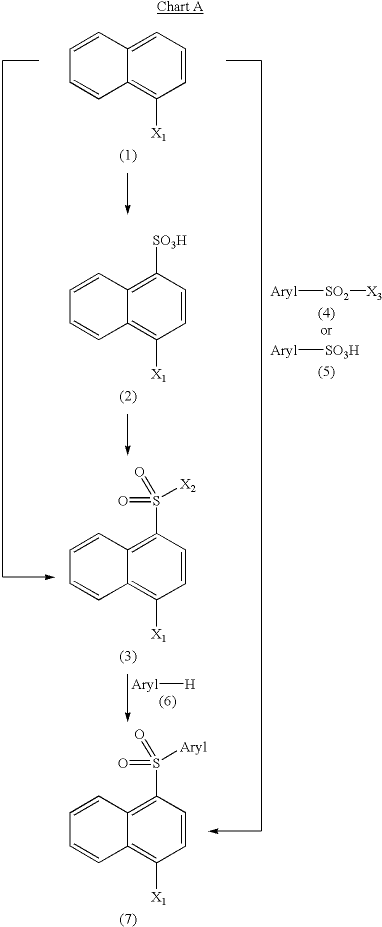

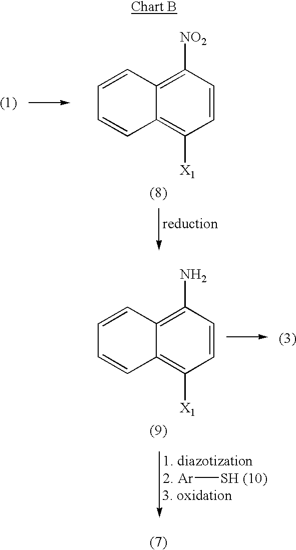

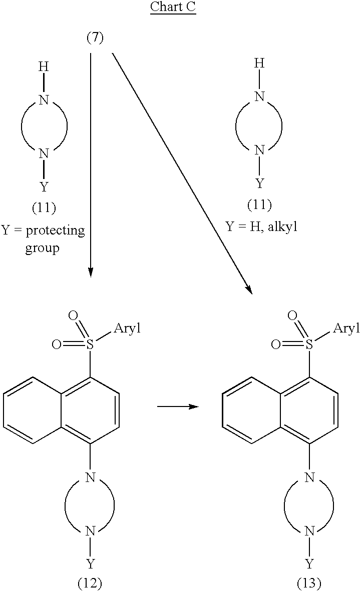

In some embodiments, the compounds are isotopically-labeled compounds. Isotopically-labeled compounds are identical to those recited in Formulae I and II, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹³N, ¹⁵O, ¹⁸F, ^{99m}Tc, ¹²³I, and ¹²⁵I. Compounds of the present invention and pharmaceutically acceptable salts and prodrugs of said compounds that contain the aforementioned isotopes and/or other isotopes of other atoms are within the scope of the invention. Isotopically-labeled compounds of the present invention are useful in drug and/or substrate tissue distribution and target occupancy assays. For example, isotopically labeled compounds are particularly useful in SPECT (single photon emission computed tomography) and in PET (positron emission tomography).

Single-photon emission computed tomography (SPECT), acquires information on the concentration of isotopically labeled compounds introduced to a mammal's body. SPECT dates from the early 1960's, when the idea of emission traverse section tomography was introduced by D. E. Kuhl and R. Q. Edwards prior to either PET, x-ray CT, or MRI. In general, SPECT requires isotopes that decay by electron capture and/or gamma emission. Example of viable SPECT isotopes include, but are not limited to, 123-iodine (¹²³I) and 99m-technetium (^{99m}Tc) . Subjects are injected with a radioactively labeled agent, typically at tracer doses. The nuclear decay resulting in the emission of a single gamma ray which passes through the tissue and is measured externally with a SPECT camera. The uptake of radioactivity reconstructed by computers as a tomogram shows tissue distribution in cross-sectional images.

Positron emission tomography (PET) is a technique for measuring the concentrations of positron-emitting isotopes within the tissues. Like SPECT, these measurements are, typically, made using PET cameras outside of the living subjects. PET can be broken down into several steps including, but not limited to, synthesizing a compound to include a positron-emitting isotope; administering the isotopically labeled compound to a mammal; and imaging the distribution of the positron activity as a function of time by emission tomography. PET is described, for example, by Alavi et al. in Positron Emission Tomography. published by Alan R. Liss, Inc. in 1985.

Positron-emitting isotopes used in PET include, but are not limited to, Carbon-11, Nitrogen-13, Oxygen-15, and Fluorine-18. In general, positron-emitting isotopes should have short half-lives to help minimize the long term radiation exposure that a patient receives from high dosages required during PET imaging.

In certain instances, PET imaging can be used to measure the binding kinetics of compounds of this invention with 5-HT₆ serotonin receptors. For example, administering an isotopically labeled compound of the invention that penetrates into the body and binds to a 5-HT₆ serotonin receptor creates a baseline PET signal which can be monitored while administering a second, different, non-isotopically labeled compound. The baseline PET signal will decrease as the non-isotopically labeled compound competes for the binding to the 5-HT₆ serotonin receptor.

In general, compounds of formula I that are useful in performing PET or SPECT are those which penetrate the blood-brain barrier, exhibit high selectivity and modest affinity to 5-HT₆ serotonin receptors, and are eventually metabolized. Compounds that are non-selective or those that exhibit excessive or small affinity for 5-HT₆ serotonin receptors are, generally, not useful in studying brain receptor binding kinetics with respect to 5-HT₆ serotonin receptors. Compounds that are not metabolized may harm the patient.

In other embodiments, nuclear magnetic resonance spectroscopy (MRS) imaging can be used to detect the overall concentration of a compound or fragment thereof containing nuclei with a specific spin. In general, the isotopes useful in NMR imaging include, but are not limited to, hydrogen-1, carbon-13, phosphorus-31, and fluorine-19. For instance, compounds containing ¹⁹F are useful in conducting NMR imaging.

Further, substitution with heavier isotopes such as deuterium, i.e., ²H, can afford certain therapeutic advantages resulting from greater metabolic stability, for example increased in vivo half-life or reduced dosage requirements and, hence, maybe preferred in some circumstances. Isotopically labeled compounds of Formula I of this invention can generally be prepared by carrying out the synthetic procedures described above by substituting an isotopically labeled reagent for a non-isotopically labeled reagent.

Compounds of the present invention can conveniently be administered in a pharmaceutical composition containing the compound in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally (for example, by intravenous, intraperitoneal or intramuscular injection), topically, orally, or rectally.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices including, but not limited to, those relying on osmotic pressures to obtain a desired release profile (e.g., the OROS drug delivery devices as designed and developed by Alza Corporation).

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. Sterilization of the powders may also be accomplished through irradiation and aseptic crystallization methods. The sterilization method selected is the choice of the skilled artisan.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. To this extent, the present invention further contemplates the use of the pharmaceutically active materials in personal care compositions such as lotions, cleansers, powders, cosmetics and the like.

The compound is conveniently administered in unit dosage form; for example, containing about 0.05 mg to about 500 mg, conveniently about 0.1 mg to about 250 mg, most conveniently, about 1 mg to about 150 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compositions can conveniently be administered orally, sublingually, transdermally, or parenterally at dose levels of about 0.01 to about 150 mg/kg, preferably about 0.1 to about 50 mg/kg, and more preferably about 0.1 to about 30 mg/kg of mammal body weight.

For parenteral administration the compounds are presented in aqueous solution in a concentration of from about 0.1 to about 10%, more preferably about 0.1 to about 7%. The solution may contain other ingredients, such as emulsifiers, antioxidants or buffers.

The exact regimen for administration of the compounds and compositions disclosed herein will necessarily be dependent upon the needs of the individual subject being treated, the type of treatment and, of course, the judgment of the attending practitioner.

Generally, compounds of the invention are 5-HT ligands. The ability of a compound of the invention to bind or act at a 5-HT receptor, or to bind or act selectively at a specific 5-HT receptor subtype can be determined using in vitro and in vivo assays that are known in the art. As used herein, the term “bind selectively” means a compound binds at least 2 times, preferably at least 10 times, and more preferably at least 50 times more readily to a given 5-HT subtype than to one or more other subtypes. Preferred compounds of the invention bind selectively to one or more 5-HT receptor subtypes.

The ability of a compound of the invention to act as a 5-HT receptor agonist or antagonist can also be determined using in vitro and in vivo assays that are known in the art. All of the Example compounds provided above are 5-HT ligands, with the ability to displace >50% of a radiolabeled test ligand from one or more 5-HT receptor subtypes at a concentration of 1 μM. The procedures used for testing such displacement are well known and illustrated below.

### 5-HT₆ RECEPORT BINDING ASSAY

Growth of Cells and Membrane Preparation

Hela cells containing the cloned human 5-HT₆ receptor were acquired from Dr. David R. Sibley's laboratory in National Institute of Health (see Sibley, D. R., J. Neurochemistry, 66, 47-56, 1996). Cells were grown in high glucose Dulbecco's modified Eagle's medium, supplemented with L-glutamine, 0.5% sodium pyruvate, 0.3% penicillin-streptomycin, 0.025% G-418 and 5% Gibco fetal bovine serum and then were harvested, when confluent, in cold phosphate buffered saline.

Harvested intact cells were washed once in cold phosphate-buffered saline. The cells were pelleted and resuspended in 100 ml of cold 50 mM Tris, 5 mM EDTA and 5 mM EGTA, pH 7.4. Homogenization was with a Vir Tishear generator, 4 cycles for 30 seconds each at setting 50. The homogenized cells were centrifuged at 700 RPM (1000 X g) for 10 minutes and the supernatant was removed. The pellet was resuspended in 100 ml of the above buffer and rehomogenized for 2 cycles. The rehomogenized cells were then centrifuged at 700 RPM (1000 X g) for 10 minutes and the supernatant was removed. The combined supernatant (200 ml) was centrifuged at 23,000 RPM (80,000 X g) for 1 hour in a Beckman Rotor (42.1 Ti). The membrane pellet was resuspended in 50-8- ml of assay buffer containing HEPES 20 mM, MgCl2 10 mM, NaCl 150 mM, EDTA 1 mM, pH 7.4 and stored frozen in aliquots at −70° C.

5-HT₆ Receptor Binding Assay

The radioligand binding assay used [³H]-lysergic acid diethylamide (LSD). The assay was carried out in Wallac 96-well sample plates by the addition of 11 μl of the test sample at the appropriate dilution (the assay employed 11 serial concentrations of samples run in duplicate), 11 μl of radioligand, and 178 μl of a washed mixture of WGA-coated SPA beads and membranes in binding buffer. The plates were shaken for about 5 minutes and then incubated at room temperature for 1 hour. The plates were then loaded into counting cassettes and counted in a Wallac MicroBeta Trilux scintillation counter.

Binding Constant (Ki) Determination

Binding Constant Determination may be obtained by performing serial dilutions, e.g., eleven dilutions, of test compounds into assay plates using the PE/Cetus Pro/Pette pipetter. These dilutions are followed by radioligand and the bead-membrane mixture prepared as described above. After obtaining the specifically bound cpm, the data are fit to a one-site binding model using GraphPad Prism ver. 2.0. Estimated IC₅₀ values are converted to Ki values using the Cheng-Prusoff equation (Cheng, Y. C. et al., Biochem. Pharmacol., 22, 3099-108, 1973).

The compounds and their preparations of the present invention will be better understood in connection with the following examples, which are intended as an illustration of and not a limitation upon the scope of the invention.

### EXAMPLES

### Preparation of 4-Fluoro-1-naphthalenesulfonyl chloride:

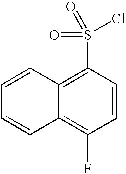

To a mixture of 1-fluoronaphthalene (1.47 g, 10.1 mmol) in chloroform (25 mL) at 0° C. was added chlorosulfonic acid (1.40 mL, 21.1 mmol) dropwise over 5-10 min. The mixture was allowed to slowly warm to room temperature while stirring overnight. The mixture was then poured onto a mixture of ice and water. The layers were separated and the aqueous layer was washed with hexane. The organic layers were combined and dried over magnesium sulfate and then concentrated to dryness under vacuum to give 2.11 g of 4-fluoro-1-naphthalenesulfonyl chloride as a white solid. ¹H NMR (CDCl₃) δ7.27, 7.77, 7.88, 8.29, 8.39, 8.80.

### Preparation of 1-Fluoro-4-(phenylsulfonyl)naphthalene:

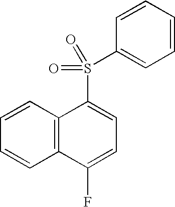

Method A. To aluminum trichloride (2.30 g, 17.2 mmol) in benzene (30 mL) was added 4-fluoro-1-naphthalenesulfonyl chloride (2.11 g, 8.62 mmol) in benzene (20 mL). The mixture was stirred at room temperature for 4 h and then poured onto a mixture of ice and water. The mixture was extracted with ethyl ether and the ether layer was washed with 1N HCl, aq. sodium bicarbonate, and brine. The organic layer was dried over magnesium sulfate and concentrated under vacuum. Crystallization from ethyl ether/hexane gave 2.10 g of 1-fluoro-4-(phenylsulfonyl)naphthalene in two crops. ¹H NMR (CDCl₃) δ7.31, 7.50, 7.62, 7.95, 8.17, 8.53, 8.62.

Method B. To a stirred mixture of aluminum trichloride (3.83 g, 28.7 mmol) in nitromethane (10 mL) was added, with cooling, 1-fluoronaphthalene (2.03 g, 13.9 mmol) in nitromethane (5 mL) over 10 min. Benzenesulfonyl chloride (2.15 g, 14.6 mmol) in nitromethane (5 mL) was added over several minutes and the mixture was allowed to warm to room temperature and stir for an additional 22 h, at which time it was poured onto ice/water and extracted with diethyl ether. The ether layer was washed with 2N HCl and brine and dried over magnesium sulfate. After concentration, the residue was crystallized from diethyl ether/hexane to give 1.28 g of 1-fluoro-4-(phenylsulfonyl)naphthalene. ¹H NMR (CDCl₃) δ7.30, 7.50, 7.64, 7.95, 8.18, 8.52, 8.63.

### Example 1

### Preparation of 1-[4-(Phenylsulfonyl)-1-naphthyl]piperazine, methane sulfonate salt via Method A:

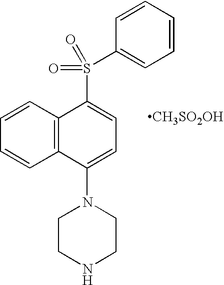

A mixture of 1-fluoro-4-(phenylsulfonyl)naphthalene (1.67 g, 5.82 mmol), piperazine (2.33 g, 27.0 mmol), and acetonitrile (15 mL) was stirred at 80° C. for 100 min and then allowed to cool. The solvent was removed under vacuum and the residue was first partitioned between ethyl ether and water, but oily solids precipitated from the mixture. The oily solids were set aside and the ether layer was washed several times with water and brine. The combined ether layers were added to the oily solids and dichloromethane was added until the solids were in solution. The mixture was dried over magnesium sulfate and concentrated under vacuum. Methanol was added and the mixture was allowed to stand. The resulting precipitate was removed by filtration and the filtrate was treated with activated charcoal. The charcoal was then removed by filtration and the filtrate was concentrated to dryness to give 1.96 g of 1-[4-(phenylsulfonyl)-1-naphthyl]piperazine. OAMS supporting ions at: ESI+353.0. ¹H NMR (CDCl₃) δ3.16, 7.12, 7.50, 7.95, 8.20, 8.45, 8.56. 1-[4-(Phenylsulfonyl)-1-naphthyl]piperazine (1.96 g, 5.56 mmol) was dissolved in methanol/dichloromethane and methanesulfonic acid (0.534 g, 5.56 mmol) was added. The solvents were removed under vacuum and the residue was crystallized from methanol/ethyl acetate to give 2.10 g of 1-[4-(phenylsulfonyl)-1-naphthyl]piperazine, methanesulfonate salt.

### Example 2

### Preparation of 1-[4-(Phenylsulfonyl)-1-naphthyl]piperazine, methanesulfonate salt via Method B:

Step 1: Preparation of 1-(4-Bromo-1-naphthyl)-2-(phenylsulfanyl)diazene

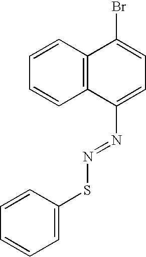

1-Amino-4-bromonaphthalene (2.57 g, 11.6 mmol) was refluxed for 5 minutes in water (50 mL) and concentrated hydrochloric acid (10 mL). The mixture was then chilled to below 5° C. in an ice/acetone bath. A solution of sodium nitrite (0.8 g, 11.6 mmol) in water (20 mL) was slowly added to the mixture, under the surface of the liquid. The mixture was stirred chilled for one hour. Sodium hydroxide (9.5 g, 237.5 mmol) was dissolved in water (100 mL). Thiophenol (1.2 mL, 11.6 mmol) was added to the hydroxide solution and the solution chilled to 5° C. using ice. The diazonium solution was slowly poured into the basic thiol solution. The mixture was allowed to stir at room temperature overnight. Solids were collected by filtration and washed with water. Column chromatography of the solids on silica gel (100 mL) using ethyl acetate:hexanes (5:95) as eluent, followed by rechromatography with silica gel (100 mL) using hexanes as eluent gave 2.67 g of the title compound; mp 57-58° C.; IR (drift) 1563, 1498, 1477, 1417, 1378, 1257, 1198, 923, 871, 812, 758, 739, 703, 685, 631 cm⁻¹. ¹H NMR (DMSO-d₆) δ7.31, 7.61, 7.78, 7.93, 7.9, 8.48.

Step 2: Preparation of 4-Bromo-1-naphthyl phenyl sulfone

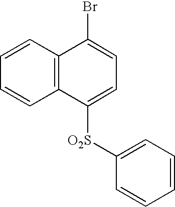

To a mixture of 1-(4-bromo-1-naphthyl)-2-(phenylsulfanyl)diazene (2.67 g, 8.47 mmol) in glacial acetic acid (50mL) was added 30% hydrogen peroxide (6.0 mL). The mixture was heated at 90° C. for 4 h. The mixture was cooled to room temperature and partitioned between water and ether. The layers were separated and the organic layer washed twice with water (200 mL). The organic layer was dried over anhydrous sodium sulfate, filtered, and concentrated. The resulting solids were triturated in methyl-t-butyl ether, collected by filtration, and dried to give 0.63 g of the title compound; IR (drift) 1499, 1308, 1200, 1153, 1139, 1084, 880, 838, 761, 751, 724, 690, 672, 625, 606 cm⁻¹. ¹H NMR (CDCl₃) δ7.5, 7.6, 7.95, 8.35, 8.65.

Step 3: Preparation of 1-[4-(Phenylsulfonyl)-1-naphthyl]piperazine, methanesulfonate salt

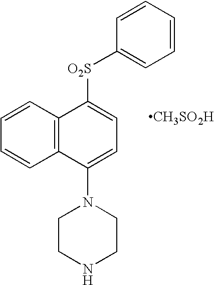

To a mixture of 4-bromo-1-naphthyl phenyl sulfone (0.59 g, 1.7 mmol) in acetonitrile (50 mL) was added potassium carbonate (0.469 g, 3.4 mmol) and piperazine (0.176 g, 2.0 mmol). The mixture was refluxed at 95° C. for 24 h. The mixture was cooled to room temperature and partitioned between water and ethyl acetate. The layers were separated and the organic layer washed three times with water (100 mL). The organic layer was dried over anhydrous sodium sulfate and concentrated. Column chromatography on silica gel (60 mL) using CH₂Cl₂:CH₃OH:NH₄OH (92:8:3) gave a solid. The solid was converted to the methanesulfonic acid salt to give 0.177 g of the title compound; mp 201-202° C.; IR (drift) 1303, 1240, 1197, 1179, 1146, 1083, 1059, 1039, 956, 785, 772, 724, 689, 619, 600 cm⁻¹. ¹H NMR (CDCl₃) δ2.88, 3.46, 3.57, 7.20, 7.49, 7.94, 8.08, 8.47, 8.61.

### Example 3

### Preparation of Cis-3,5-Dimethyl-1-[4-(phenylsulfonyl)-1-naphthyl]piperazine:

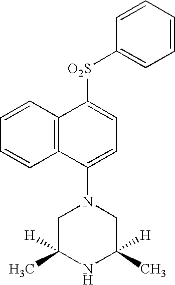

To a mixture of 1-fluoro-4-(phenylsulfonyl)naphthalene (0.45 g, 1.57 mmol) in acetonitrile (20 mL) was added potassium carbonate (0.745 g, 5.4 mmol) and cis-2,5-dimethyl piperazine (0.536 g, 4.7 mmol). The mixture was refluxed at 90° C. overnight. The mixture was partitioned between water and ethyl acetate. The layers were separated and the organic layer washed twice with water (50 mL). The organic layer was dried over anhydrous magnesium sulfate and concentrated. Column chromatography on silica gel (50 mL) using methanol/dichloromethane (5/95) gave 0.267 g of the title compound; mp 168-169° C.; IR (drift) 2964, 2959, 1568, 1508, 1320, 1302, 1195, 1152, 1140, 1084, 1061, 767, 723, 686, 669 cm⁻¹. ¹H NMR (CDCl₃) δ1.12, 1.15, 2.47, 3.27, 3.33, 7.09, 7.5, 7.93, 8.16, 8.43, 8.55.

### Example 4

### Preparation of 1-[4-(Phenylsulfonyl)-1-naphthyl]-1,4-diazepane, methanesulfonate salt

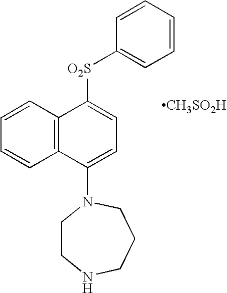

To a mixture of 1-fluoro-4-(phenylsulfonyl)naphthalene (0.45 g, 1.6 mmol) in acetonitrile (25 mL) was added homopiperazine (0.258 g, 6.8 mmol) and potassium carbonate (0.47 g, 4.8 mmol). The mixture was refluxed at 90° C. overnight. The mixture was partitioned between water and ethyl acetate. The layers were separated and the organic layer washed twice with water (100 mL). The organic layer was dried over anhydrous magnesium sulfate, filtered and concentrated. Column chromatography on silica gel (75 mL) using methanol/dichloromethane (5/95) and conversion to the methanesulfonic acid salt gave 0.069 g of the title compound; mp 110-111° C.; IR (drift) 3007, 2985 (b), 2957 (b), 2935 (b), 2831 (b), 2778 (b), 2353, 2339 (w), 1995 (w), 1990 (w), 1965 (w), 1197 (s), 1180, 1152 (s), 724 (s), cm⁻¹. ¹H NMR (CDCl₃) δ2.36, 3.4, 3.6, 3.6, 7.24, 7.5, 7.93, 8.2, 8.44, 8.6.

### Example 5

### Preparation of 1-{4-[(2,5-Dimethylphenyl)sulfonyl]-1-naphthyl}piperazine, methanesulfonate salt:

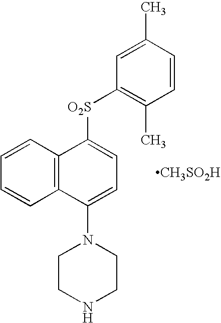

Step 1: Preparation of 1-[(2,5-Dimethylphenyl)sulfonyl]-4-fluoronaphthalene:

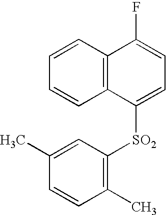

To a mixture of 1-fluoronaphthalene (1.05 g, 7.18 mmol) and p-xylene-2-sulfonyl chloride (1.47 g, 7.18 mmol) in nitromethane (10 mL) was added aluminum trichloride (1.9 g, 14.4 mmol) in portions over 1 minute. The mixture was stirred at room temperature overnight. The mixture was partitioned between water and ethyl acetate. The layers were separated and the organic layer washed twice with water (50 mL). The organic layer was dried over anhydrous magnesium sulfate and concentrated. The resulting solids were slurried in Et₂O, collected by filtration, and dried to give 1.18 g of the title compound; mp 142-143° C.; ¹H NMR (CDCl₃) δ2.3, 2.44, 7.05, 7.3, 7.6, 8.15, 8.18, 8.44.

Step 2: Preparation of 1-{4-[(2,5-Dimethylphenyl)sulfonyl]-1-naphthyl}piperazine, methanesulfonate salt

To a mixture of 1-[(2,5-dimethylphenyl)sulfonyl]-4-fluoronaphthalene (0.6 g, 1.9 mmol) in acetonitrile (20 mL) was added potassium carbonate (1.58 g, 11.4 mmol) and piperazine (0.82 g, 9.5 mmol). The mixture was refluxed at 90° C. overnight. The mixture was partitioned between water and ethyl acetate. The layers were separated and the organic layer washed twice with water (50 mL). The organic layer was dried over anhydrous magnesium sulfate and concentrated. Column chromatography on silica gel (100 mL) using 4% CH₃OH/CH₂Cl₂ (4/96) and conversion to the methanesulfonic acid salt gave 0.0769 g of the title compound; ¹H NMR (CDCl₃) δ2.29, 2.44, 3.54, 7.05, 7.24, 7.55, 8.07, 8.15, 8.39, 8.47.

### Example 6

### Preparation of 4-Methylphenyl 4-(1-piperazinyl)-1-naphthyl sulfone:

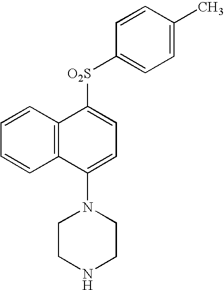

Step 1: Preparation of 1-Fluoro-4-[(4-methylphenyl)sulfonyl]naphthalene:

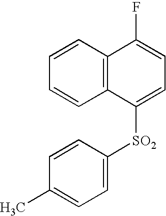

To a mixture of p-toluenesulfonyl chloride (1.31 g, 6.9 mmol) in nitromethane (10 mL) was added 1-fluoronaphthalene (1.01 g, 6.9 mmol). To the mixture was added aluminum trichloride (1.94 g, 14.5 mmol) in portions. The mixture was stirred at room temperature overnight. The mixture was partitioned between water and ethyl acetate. The layers were separated and the organic layer washed twice with water (50 mL). The organic layer was dried over anhydrous magnesium sulfate and concentrated. Column chromatography on silica gel (60 mL) using ethyl acetate:hexanes (10:90) gave 0.182 g of the title compound; ¹H NMR (CDCl₃) δ2.36, 7.26, 7.62, 7.82, 8.16, 8.5, 8.6.

Step 2: Preparation of 4-Methylphenyl 4-(1-piperazinyl)-1-naphthyl sulfone:

To a mixture of 1-fluoro-4-[(4-methylphenyl)sulfonyl]naphthalene (0.157 g, 0.52 mmol) in acetonitrile (10 mL) was added potassium carbonate (0.43 g, 3.2 mmol) and piperazine (0.224 g, 2.6 mmol). The mixture was refluxed at 90° C. overnight. The mixture was partitioned between water and ethyl acetate. The layers were separated and the organic layer washed twice with water (50 mL). The organic layer was dried over anhydrous magnesium sulfate and concentrated. Column chromatography on silica gel (50 mL) using CH₃OH:CH₂Cl₂ (5:95) gave 0.0297 g of the title compound; ¹H NMR (CDCl₃) δ2.35, 3.16, 7.09, 7.23, 7.5, 7.8, 8.18, 8.42, 8.57.

### Example 7

### Preparation of 4-(4-Methyl-1-piperazinyl)-1-naphthyl phenyl sulfone:

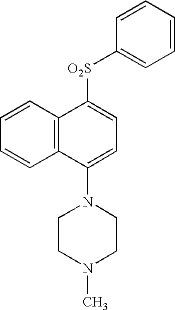

To a mixture of 1-fluoro-4-(phenylsulfonyl)naphthalene (0.45 g, 1.57 mmol) in acetonitrile (20 mL) was added potassium carbonate (0.54 g, 3.9 mmol) and N-methyl piperazine (0.35 g, 3.5 mmol). The mixture was refluxed at 90° C. overnight. The mixture was partitioned between water and ethyl acetate. The layers were separated and the organic layer washed twice with water (100 mL). The organic layer was dried over anhydrous magnesium sulfate and concentrated. Column chromatography on silica gel (50 mL) using CH₃OH:CH₂Cl₂ (5:95) gave 0.069 g of a solid. The solid was dissolved in CH₂Cl₂ and activated charcoal (0.4 g) was added. The mixture was stirred at room temperature for 1 hour. The mixture was filtered through diatomaceous earth and concentrated to give 0.029 g of the title compound; mp 71-72° C.; ¹H NMR (CDCl₃) δ2.42, 2.72, 3.22, 7.11, 7.5, 7.93, 8.16, 8.43, 8.55.

### Example 8

Utilizing the procedure of Example 1 and substituting the appropriately substituted isoquinoline starting material for 1-fluoro-4-(phenylsulfonyl)naphthalene, there is obtained 4-(phenylsulfonyl)-1-piperazine-1-ylisoquinoline.

### Example 9

Utilizing the procedure of Example 1 and substituting the appropriately substituted isoquinoline starting material for 1-fluoro-4-(phenylsulfonyl)naphthalene, there is obtained 1-(phenylsulfonyl)-4-piperazine-1-ylisoquinoline.

### Example 10

Utilizing the procedure of Example 1 and substituting the appropriately substituted phthalazine starting method for 1-fluoro-4-(phenylsulfonyl)naphthalene, there is obtained 1-(phenylsulfonyl)-4-piperazine-1-ylphthalazine.

What is claimed is:

1. A compound of formula I: 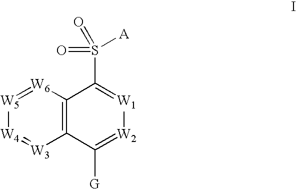
or a pharmaceutically acceptable salt thereof, wherein
Each of W₁-W₆ are independently N or —C(R₁), provided that no more than three of W₁-W₆are simultaneously N, and further provided that when W₁ is N that W₂ is not —CHaryl, or —CHaryl in which the aryl group is substituted with halo, —OH, —CN, —NO₂, —CF₃, —COOR₁, tetrazolyl, or isoxazolyl;
Each R₁ is independently selected from H, halo, alkyl, cycloalkyl, substituted alkyl, —OH, alkoxy, substituted alkoxy, —SH, —S-alkyl, —S-substituted alkyl, —CN, —NO₂, —NR₄R₅, —NR₄SO₂-alkyl, —NR₄SO₂-aryl, —COOR₄, —CONR₄R₅, —SO₂NR₄R₅, —SO₂-alkyl, het, substituted het, aryl, and substituted aryl;
Each R₄ and R₅ is independently H, alkyl, cycloalkyl, substituted alkyl, aryl, het, substituted aryl, or substituted het, or R₄ and R₅ when taken together form a five, six, or seven-membered ring which contains 1-3 heteroatoms selected from N, O, or S;
A is a five- or six-membered monocyclic aromatic ring; a eight- or ten-membered fused aromatic ring, the five- or six-membered monocyclic aromatic ring and the eight- or ten-membered fused aromatic ring system each optionally containing up to three heteroatoms (O, N, S); or a nine-membered fused aromatic ring system containing one to three heteroatoms (O, N, S), and each of the five- or six-membered monocyclic aromatic ring and the eight- to ten-membered fused aromatic ring systems being optionally substituted with 1-4 of R₁, and when all of W₁-W₆ are —(CH)R₁ A is substituted with at least one electron donating group;
G is a group selected from 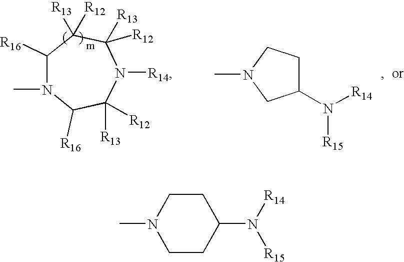
Each R₁₂ and R₁₆ is independently selected from H, alkyl, and oxo, provided that R₁₃ is absent when the oxo moiety is bound to the same carbon;
Each R₁₃ is H or alkyl;
Each R₁₄ and R₁₅ is independently H, alkyl, and substituted alkyl; and
m is 0 or 1.

2. A compound of claim 1, wherein each R₁ is independently selected from H, halo, C₁-C₆ alkyl, C₃-C₇ cycloalkyl, C₁-C₃ alkyl-C₃-C₇-cycloalkyl, —CF₃, —OH, —O—(C₁-C₆-alkyl) , —O—C₂-C₆-alkyl—OH, —O—C₂-C₆-alkyl-NR₂R₃, —OCF₃, —SH, —S—(C₁-C₆-alkyl) , —CN, —NO₂, —NR₄R₅, —NHSO₂-C₁-C₄-alkyl, —COOR₄, —CONR₄R₅, —SO₂NR₄R₅, —SO₂-C₁-C₄-alkyl, and aryl optionally substituted with H, halo, C₁-C₆-alkyl, C₁-C₆-cycloalkyl, —OH, —O—(C₁-C₆-alkyl), —CN, —NR₄R₅, —CONR₄R₅, or —SO₂NR₄R₅.

3. A compound of claim 2, wherein each R₂ and R₃ is independently H or C₁-C₄-alkyl.

4. A compound of claim 1, wherein each R₄ and R₅ is independently H, C₁-C₄-alkyl, C₃-C₇-cycloalkyl, or C₁-C₃-alkyl-C₃-C₇-cycloalkyl.

5. A compound of claim 1, wherein each R₁₄ and R₁₅ is independently H, C₁-C₆-alkyl, or C₂-C₄-alkyl—OH.

6. A compound of claim 3, wherein each R₄ and R₅ is independently H, C₁-C₄-alkyl, C₃-C₇-cycloalkyl, or C₁-C₃-alkyl-C₃-C₇-cycloalkyl.

7. A compound of claim 6, wherein each R₁₄ and R₁₅ is independently H, C₁-C₆-alkyl, or C₂-C₄-alkyl—OH.

8. A compound of claim 1, wherein G is

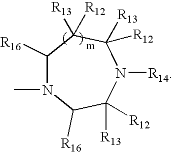

9. A compound of claim 8, wherein m is 0.

10. A compound of claim 8, wherein R₁₄ is —CH₃.

11. A compound of claim 8, wherein each R₁₂ is —CH₃.

12. A compound of claim 8, wherein m is 1.

13. A compound of claim 1, wherein A is substituted with the electron donating group and one R₁, the R₁ being —CH₃.

14. A compound of claim 1, wherein A is substituted with the electron donating group and two R₁ groups, both of the R₁ groups being —CH₃.

15. A compound of claim 1, wherein all of W₁-W₆ are —C(R₁).

16. A compound of claim 1, wherein at least one of W₁-W₆ is N

17. A compound of claim 16, wherein G is

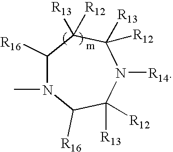

18. A compound of claim 17, wherein m is 0.

19. A compound of claim 17, wherein R₁₄ is —CH₃.

20. A compound of claim 17, wherein each R₁₂ is —CH₃.

21. A compound of claim 17, wherein m is 1.

22. A compound of claim 16, wherein A is substituted with one R₁, the R₁ being —CH₃.

23. A compound of claim 16, wherein A is substituted with two R₁ groups, both of the R₁ groups being —CH₃.

24. A compound of claim 1 selected from the group consisting of
1-[4-(Phenylsulfonyl)-1-naphthyl]piperazine;
Cis-3,5-Dimethyl-1-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

25. A pharmaceutical composition comprising a therapeutically effective amount of a compound of formula I or a pharmaceutically acceptable salt thereof.

26. A pharmaceutical compound of claim 25, wherein the compound is selected from the group consisting of 1-[4-(Phenylsulfonyl)-1-naphthyl]piperazine;
Cis-3,5-Dimethyl-1-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

27. A pharmaceutical composition of claim 24, wherein the composition further comprises a pharmaceutically acceptable carrier.

28. A method for treating a disease or condition in a mammal wherein a 5-HT receptor is implicated and modulation of a 5-HT function is desired comprising administering to the mammal a therapeutically effective amount of a compound, or pharmaceutically acceptable salt thereof, of formula I or II: 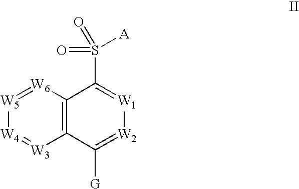
wherein
Each of W₁-W₆ are independently N or —C(R₁), provided that no more than three of W₁-W₆are simultaneously N;
Each R₁ is independently selected from H, halo, alkyl, cycloalkyl, substituted alkyl, —OH, alkoxy, substituted alkoxy, —SH, —S-alkyl, —S-substituted alkyl, —CN, —NO₂, —NR₄R₅, —NR₄SO₂-alkyl, —NR₄SO₂-aryl, —COOR₄, —CONR₄R₅, —SO₂NR₄R₅, —SO₂-alkyl, het, substituted het, aryl, and substituted aryl;
Each R₄ and R₅ is independently H, alkyl, cycloalkyl, substituted alkyl, aryl, het, substituted aryl, or substituted het, or R₄ and R₅ when taken together form a five, six, or seven-membered ring which contains 1-3 heteroatoms selected from N, O, or S;
A is a five- or six-membered monocyclic aromatic ring; a eight- or ten-membered fused aromatic ring, the five- or six-membered monocyclic aromatic ring and the eight- or ten-membered fused aromatic ring system each optionally containing up to three heteroatoms (O, N, S); or a nine-membered fused aromatic ring system containing one to three heteroatoms (O, N, S), and each of the five- or six-membered monocyclic aromatic ring and the eight- to ten-membered fused aromatic ring systems being optionally substituted with 1-4 of R₁;
G is a group selected from 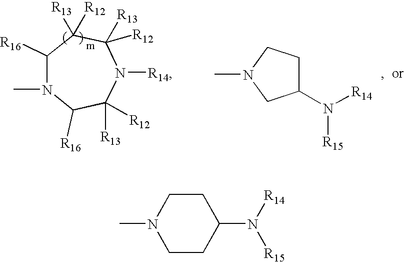
Each R₁₂ and R₁₆ is independently selected from H, alkyl, and oxo, provided that R₁₃ is absent when the oxo moiety is bound to the same carbon;
Each R₁₃ is H or alkyl;
Each R₁₄ and R₁₅ is independently H, alkyl, and substituted alkyl; and
m is 0 or 1.

29. The method of claim 28, wherein each R₁ is independently selected from H, halo, C₁-C₆ alkyl, C₃-C₇ cycloalkyl, C₁-C₃ alkyl-C₃-C₇-cycloalkyl, —CF₃, —OH, —O—(C₁-C₆-alkyl), —O—C₂-C₆-alkyl—OH, —O—C₂-C₆-alkyl-NR₂R₃, —OCF₃, —SH, —S—(C₁-C₆-alkyl), —CN, —NO₂, —NR₄R₅, —NHSO₂-C₁-C₄-alkyl, —COOR₄, —CONR₄R₅, —SO₂NR₄R₅, —SO₂-C₁-C₄-alkyl, and aryl optionally substituted with H, halo, C₁-C₆-alkyl, C₁-C₆-cycloalkyl, —OH, —O—(C₁-C₆-alkyl), —CN, —NR₄R₅, —CONR₄R₅, or —SO₂NR₄R₅.

30. The method of claim 29, wherein each R₂ and R₃ is independently H or C₁-C₄-alkyl.

31. The method of claim 28, wherein each R₄ and R₅ is independently H, C₁-C₄-alkyl, C₃-C₇-cycloalkyl, or C₁-C₃-alkyl-C₃-C₇-cycloalkyl.

32. The method of claim 28, wherein each R₁₄ and R₁₅ is independently H, C₁-C₆-alkyl, or C₂-C₄-alkyl—OH.

33. The method of claim 30, wherein each R₄ and R₅ is independently H, C₁-C₄-alkyl, C₃-C₇-cycloalkyl, or C₁-C₃-alkyl-C₃-C₇-cycloalkyl.

34. The method of claim 33, wherein each R₁₄ and R₁₅ is independently H, C₁-C₆-alkyl, or C₂-C₄-alkyl—OH.

35. The method of claim 28, wherein G is

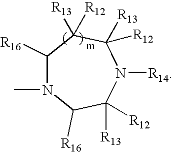

36. The method of claim 35, wherein m is 0.

37. The method of claim 35, wherein R₁₄ is —CH₃.

38. The method of claim 35, wherein each R₁₂ is —CH₃.

39. The method of claim 35, wherein m is 1.

40. The method of claim 28, wherein A is substituted with one R₁, the R₁ being —CH₃.

41. The method of claim 28, wherein A is substituted with two R₁ groups, both of the R₁ groups being —CH₃.

42. The method of claim 28, wherein all of W₁-W₆ are —C(R₁).

43. The method of claim 28, wherein the compound is 1-[4-((Phenylsulfonyl)-1-naphthyl]piperazine;
Cis-3,5-Dimethyl-1-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

44. A method for treating a disease or condition in a mammal wherein a 5-HT₆ receptor is implicated and modulation of a 5-HT₆ function is desired comprising administering to the mammal a therapeutically effective amount of a compound of formula I or II, or a pharmaceutically acceptable salt thereof.

45. A compound of formulae I or II, wherein the compound includes an isotopic label.

46. The compound of claim 45, wherein the compound includes at least on atom selected from Carbon-11, Nitrogen-13, Oxygen-15, and Fluorine-18.

47. A compound of claim 45, wherein the compound is 1-[4-(Phenylsulfonyl)-1-naphthyl]piperazine;
Cis-3,5-Dimethyl-l-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

48. A method of performing positron emission tomography comprising incorporating an isotopically labeled compound of formula I or II or a pharmaceutically acceptable salt thereof into tissue of a mammal and detecting the compound distributed in said tissue.

49. A method of claim 48, wherein the compound includes at least one atom selected from Carbon-11, Nitrogen-13, Oxygen-15 and Fluorine 18.

50. A method of claim 48, wherein the compound is 1-[4-(Phenylsulfonyl)-1-naphthyl]piperazine;
Cis-3,5-Dimethyl-1-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

51. A method of claim 48, wherein the mammal is a human.

52. A method of performing nuclear magnetic resonance imaging comprising incorporating an isotopically labeled compound of formula I or II or a pharmaceutically acceptable salt thereof into tissue of a mammal and detecting the compound distributed in said tissue.

53. A method of claim 52, wherein the compound includes at least one Fluorine-19 atom.

54. A method of claim 52, wherein the compound is 1-[4-(Phenylsulfonyl)-1-naphthyl]piperazine;
Cis-3,5-Dimethyl-1-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

55. A method of claim 52, wherein the mammal is a human.

56. A method of performing single photon emission computed tomography comprising incorporating an isotopically labeled compound of formula I or II or a pharmaceutically acceptable salt thereof into tissue of a mammal and detecting the compound distributed in said tissue.

57. A method of claim 56, wherein the compound includes at least one atom selected from Iodine-123 or 99m-technetium.

58. A method of claim 56 wherein the compound is 1-[4-(Phenylsulfonyl)-1-naphthyl]piperazine;
Cis-3,5-Dimethyl-1-[4-(phenylsulfonyl)-1-naphthyl]piperazine;
1-[4-(Phenylsulfonyl)-1-naphthyl]-1,4-diazepane;
1-{4-[(2,5-Dimethylphenyl)sulfonyl]-1-naphthyl}piperazine;
4-Methylphenyl 4-(1-piperazinyl)-1-naphthyl sulfone;
4-(4-Methyl-1-piperazinyl)-1-naphthyl phenyl sulfone;
or a pharmaceutically acceptable salt thereof.

59. A method of claim 56, wherein the mammal is a human.
